(19) United States
(12) Patent Application Publication
(10) Pub. No.: US 2022/0199249 A1
(43) Pub. Date: Jun. 23, 2022

(54) SYSTEM AND METHOD TO SELF-DETERMINE A MENTAL AND EMOTIONAL (NON-PHYSICAL) WELLNESS SCORE OVER TIME USING DEEP LEARNING ALGORITHMS (BASED ON COGNITIVE BIAS) WHICH RESPOND TO VARIOUS ACTIVITIES AND EVENTS THROUGH A SERIES OF SENSORS, FEEDBACK, ACTIVITIES AND CONVERSATIONAL METHODS.

(71) Applicants: Jeffrey O'Dell, Berlin, MA (US); Mahalingam Kamalahasan, Springfield, MA (US)

(72) Inventors: Jeffrey O'Dell, Berlin, MA (US); Mahalingam Kamalahasan, Springfield, MA (US)

(21) Appl. No.: 17/247,742

(22) Filed: Dec. 21, 2020

(51) Int. Cl. G16H 40/67; G06N 3/08; G16H 20/70

(52) U.S. Cl. CPC G16H 40/67; G16H 20/70; G06N 3/08

(57) ABSTRACT

The vast majority of emphasis is still on physical health such as diet and exercise. Although some progress has been made in mental health, a new way of wellness can be achieved by extending the mental health space beyond relaxation techniques such as meditation. This is done by calculating a wellness score once we understand the cognitive/psychological bias of a person and knowing their past and present activities, events and their experiences. This wellness score is used as a basis to improve the emotional and mental health of the individual by recommending Next Best Actions (NBA.) NBA could include several activities that the user could perform beyond current relaxation methods. The pursuit of wellness obtained through this holistic approach proves it to be highly effective to address mental and emotional health rather than one solution fits all approaches that we see in the mental health industry.

### BACKGROUND

### Field of the Invention

The invention generally relates to System and Methods to self-determine a mental and emotional (non-physical) wellness score over time using deep learning algorithms (based on cognitive bias) which respond to various activities and events through a series of sensors, feedback, activities and conversational methods.

### Background

The vast majority of the wellness system has emphasis on physical activities such as diet and exercise. The non-physical activities in the existing systems are limited to relaxation techniques such as sounds, meditation and yoga; they lack any understanding of the emotional, mental and spiritual health of an user, a serious limitation. In addition to this, there is no system that deals with wellness by holistically combining physical, emotional, mental and spiritual health of an individual. Because there is no existing system, there is no method that collects such holistic data from the user. With no existing system and methods, we learned that a wellness scoring engine with an overall wellness score for improving the mental, emotional and spiritual health has never been developed.

### SUMMARY OF THE INVENTION

The problems outlined above are in large measure solved by this present invention. The system disclosed herein provides means to assist the user to maintain their wellness by tracking their emotional and mental health and providing next best actions the user needs to perform to improve their overall well-being. The net wellness score at a point in time and continuous trend graphed in a time scale is a critical feedback system to improve and maintain the overall wellness. The net score is calculated by the scoring engine which comprises deep learning algorithms. Deep learning algorithms in this invention are built based on the user's cognitive biases.

### BRIEF DESCRIPTION OF THE DRAWINGS

FIG. 1 is a schematic view of a system in accordance with the present invention;

FIG. 2 is a logical flow diagram showing the overall operation of the present invention;

FIG. 3 is a flowchart depicting in greater detail the steps to determine the color band for the user based on the calculated wellness score;

FIG. 4 is a flowchart depicting in greater detail the steps to calculate user wellness score using non-bias method when the user logs in to system for the very first time;

FIG. 5 is a flowchart provides steps in detail on how cognitive bias is applied in the algorithm to calculate the wellness score;

FIG. 6 is a flowchart depicting in greater detail on how regularization formula is applied for negativity bias to calculate the wellness score;

FIG. 7 is a flowchart depicting in greater detail on how confirmation bias is applied in the algorithm to calculate the wellness score;

FIG. 8 is a flowchart depicting in greater detail on how the wellness score is calculated from the sleep data retrieved from the wearable device;

FIG. 9 is a flowchart depicting in greater detail the steps performed by the algorithm to calculate the wellness score based on the outcome from user's activity;

### DETAILED DESCRIPTION OF THE DRAWINGS

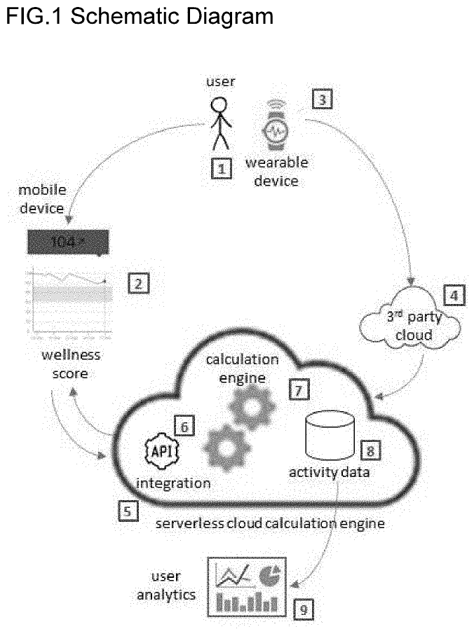

Referring to the Wellness Score Schematic diagram in FIG. 1, a system that comprises mobile application 2, wearable device 3, cloud computing resources 5 to calculate the wellness score for the user. Also includes a reporting tool 9 which provides insights into user's wellness.

To further explain the operation, user 1 signs in to the mobile application 2 on a daily basis. The user 1 answers questions, shares life events, updates their daily activities, records a journal of their dreams in the mobile application 2. At the same time sleep data from the user's wearable device 3 is fed to the 3rd party cloud 4 on a frequent basis. The user data collected from mobile application 2 and wearable device 3 (via 3rd party cloud 4) is then fed to the on-demand computing resources called Wellness Score Calculation Engine Cloud 5 (also simply known as cloud).

The wellness score calculation engine cloud 5 comprises Application Programming Interface (API) 6, calculation engine 7 with deep learning algorithms and a database 8 to collect and store user's profile, activity, sleep and event data. The calculation engine 7 applies its deep learning algorithm on the user's data stored in database 8 to come up with a wellness score. The user 1 signed in to the mobile application 2 uses API 6 to get their wellness score. User Reporting tool 9 collects data from the database 8 and provides insights into the user's well-being.

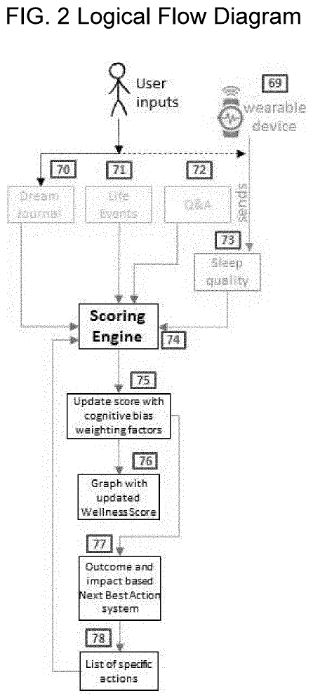

Referring to FIG. 2, is a logical flow diagram that shows the overall operation of the present invention. The user after signing in to the mobile application 2 journals their dream in the Dream Journal 70, responds to daily, weekly and monthly questions generated by the Q&A module 72, and responds to questions related to life events (e.g. marriage, new born baby, loss in family etc.,) generated by the Life Events module 71. Sleep Quality data 73 is also collected from user's wearable device 69. User's completed actions based on the List of Specific Actions module 78 are also collected. The Scoring Engine 74 applies the deep learning algorithms to the data collected from the Dream Journal 70, Q&A module 72, Life Events module 71 and Sleep Quality data module 73, List of Specific Actions module 74 to come up with a wellness score for the user. Cognitivity bias weighting 75 is applied to the user's wellness score to come up with the user's Net Wellness Score. The graph 76 in the mobile application 2 (refer FIG. 1) is then updated with this Net Wellness Score. The user's Net Wellness Score is also fed to Outcome and Impact—Next Best Action module 77. The Next Best Action module 77 is used as foundation to determine the next best action recommendation for the user. This is done so that the user can achieve the expected outcome with high impact. The information from Next Best Action module 77 is then fed to List of Specific Actions module 78. The user completes actions listed in the List of Specific Actions module 78 and results are fed back into the Scoring Engine 74. This process continues and feeds itself with more data. With continuous feeding of data from the user and regular feedback from the user this system constantly learns and improves the recommendation and overall effectiveness for the user. The above steps completes the overall steps and operation at a high level for the present invention.

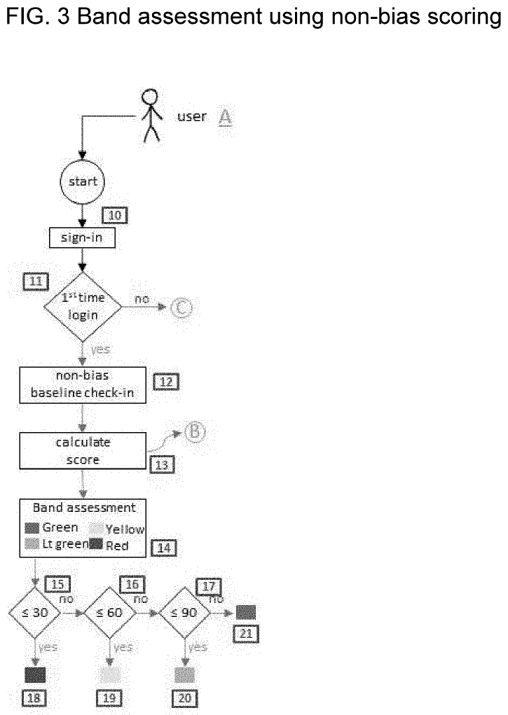

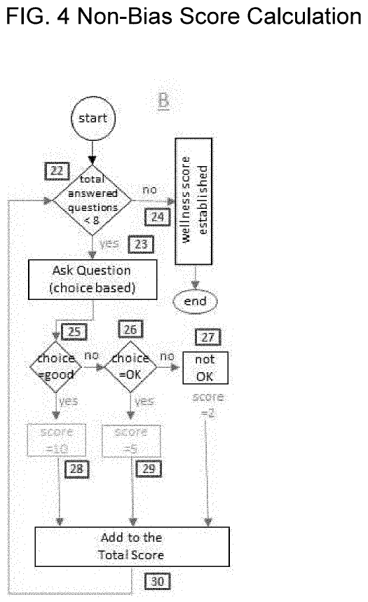

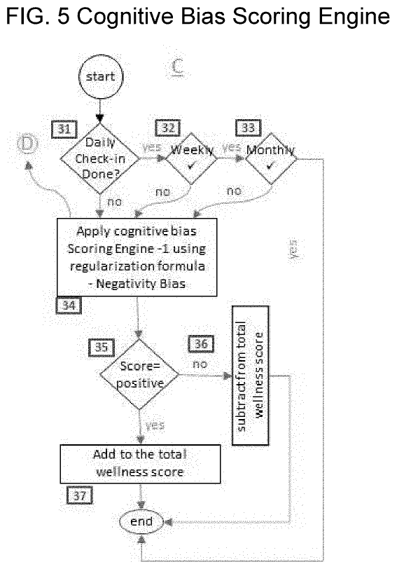

Referring to FIG. 3, is a flowchart A representing one of the scoring components built in the Scoring Engine 75 mentioned in FIG. 2. This scoring component assigns a baseline color for the user also known as color band. The steps followed to assign a color band to the user is explained in this section. User signs-in 10 to the mobile application 2 (refer FIG. 1). Determine if the user is signing in to the application for the first time using 1st Time Login module 11. If the user is not signing in for the first time then redirect the user to the steps implemented as part of the flow chart C mentioned in FIG. 5. In the case of first time signed in users, the users are assigned eight baseline questions generated by the Non-Bias Baseline Check-in module 12. To make sure that the user has answered all eight questions the steps mentioned in flow chart B referred in FIG. 4 is executed first. After receiving responses for all the eight questions the user's answers are then assessed and a score is calculated using the module Calculate Score 13. The calculated score is then fed to the Band Assessment module 14. If the users score is less than or equal to thirty 15, the user is assigned a red color band 18. If the user's score is less than or equal to sixty but greater than thirty 16, the user is assigned a yellow color band 19. If the user's score is less than or equal to ninety but greater than sixty, the user is assigned a light green band 20. If the user's score is greater than ninety, the user is assigned a green band 21.

Referring to FIG. 4, is a flowchart B describing the steps used to calculate non-bias score for all users signed in for the first time. This component is built in the Calculate Score module 13 mentioned in FIG. 3. This flow chart details the steps needed to calculate the baseline wellness score for the user. The steps followed are explained in detail in this section. Step 22 checks if the user has answered less than eight questions. In case if the user has answered all eight questions, Wellness Score Established event 24 is created. Otherwise, the user is asked to respond to choice based questions 23. The Ask Question module 23 is executed for every question that the user must answer. Every question in the Ask Question module 23 has three choices. They are Good 25, OK 26, and Not OK 27. In the event when the user chooses Good 25, the user is awarded a score of ten points and added to the user's total score 30. In the event when the user chooses OK 26, the user is awarded a score of five points and added to the user's total score 30. In the event when the user chooses Not OK 27, the user is awarded a score of two points and added to the total score 30. This process is repeated until all eight questions 22 are answered by the user.

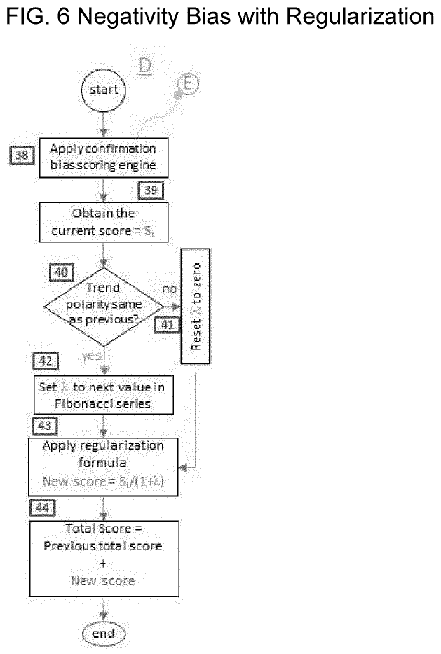

Referring to FIG. 5, is a flowchart C explaining how negativity bias is applied to user's experience. Negativity bias is one part of Cognitive Bias in which more psychological weight is given to a user's bad experiences when the user in general maintains good mental and emotional health. Similarly less psychological weight is given to a positive experience felt when the user in general maintains good mental and emotional health. Converse is also true, i.e. less psychological weight is given to a user's negative experience when the user in general does not maintain good mental and emotional health. Similarly more psychological weight is given to a positive experience felt when the user in general does not have good mental and emotional health. Mathematically psychological weight is achieved through a regularization formula and the amount (more or less) is achieved through the polarity (positive or negative) to the calculated score. This flowchart C explains in detail the steps performed. The user after signing in to the mobile application 2 referred in FIG. 1, needs to report activities by answering daily 31, weekly 32 and monthly 33 check-in questions. The Daily Check-in module 31 presents the user with daily check-in questions if they have not answered them earlier. The answers received from the user are then fed to Regularization module 34. In case if the user has already answered daily check-in questions, the weekly check-in module 32 presents the user with weekly check-in questions if they have not answered those questions earlier for that week. The answers received from the user are then fed to Regularization module 34. In case if the user has already answered weekly check-in questions, the monthly check-in module 33 presents the user with monthly check-in questions if they have not answered those questions earlier for that month. The answers received from the user are then fed to Regularization module 34. In case if the user has already answered monthly check-in questions, no other wellness score calculation is needed at this point for this user. The score received from the Regularization module 34 is checked for its polarity 35. If the score is positive, the calculated score is added to the user's total wellness score 37. Else, the calculated score is subtracted from the user's total wellness score 36. The Regularization module 34 is explained in detail in flowchart D referred in FIG. 6.

Referring to FIG. 6, is a flowchart D explaining in detail the regularization formula applied in the Cognitive Bias Scoring Engine 34 referred to in flowchart C. The flowchart D explains in details the steps performed. The user's data is fed to the confirmation bias scoring engine 38. The current score (Sₜ) for the user is obtained in step 39. The trend polarity is obtained by knowing the outcome of the current event for the user. If the outcome resulted in a positive experience for the user then the trend polarity for the event is positive. If the outcome resulted in a negative experience for the user then the trend polarity for the event is negative. If the user's trend polarity is same as the previous one reported by the user 40, the current lambda (λ) value for the user is retrieved and set to the next value in the fibonacci series 42. The score Sₜ and lambda (λ) are then used in the regularization formula 43 to obtain new score for the event. If the user's trend polarity is not the same as the previous one reported by the user 40 then lambda (λ) is reset to zero 41. This new lambda (λ) value along with the score Sₜ is used in the regularization formula 43 to obtain new score for the event. The detailed explanation of the regularization formula 43 is as follows;

Sₜ/(1+λₜₙ)

S is the absolute score of the current event (no polarity)

t is the trend polarity of the event, positive or negative trend for Score S.

Sₜ is the score of the current event with the trend polarity. I.e. if the user continues to report events of bad experience then the trend is negative.

n is the number of consecutive number of events of type t in a given period y.

y is the epoch, period in weeks in general. For Sleep activity the period is in days.

Note: y is critical to the epoch, i.e to determine if the events are relatively current and if they are applicable to calculate the score.

y will be used to determine if the user needs to take a re-baseline. For example, if the user has not logged in to the application for the last six months, the new baseline would help to calculate the score that is more appropriate to the current standing of the person, in this scenario the cumulative score C resets to 0.

C is the cumulative score for a category.

Category is classified into four types.
1. Checkin,
2. Sleep,
3. Dream, and
4. Actions/Goals

λ will be greater than zero only when the trend polarity of the events and polarity of cumulative score are the same.

The new score obtained from the regularization formula 43 is then added to the cumulative score to obtain a new cumulative score 44 for that category C.

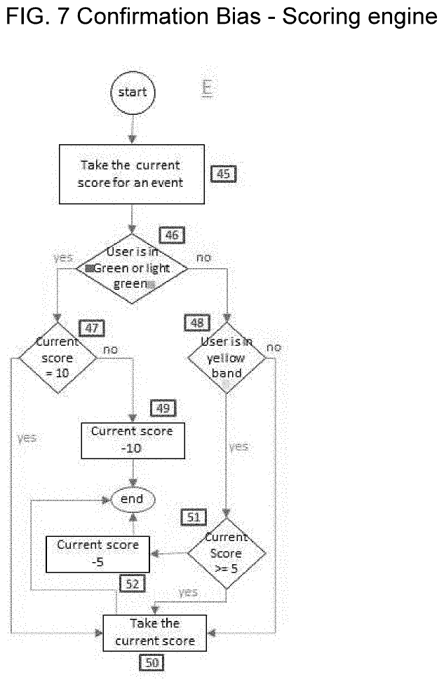

Referring to FIG. 7, is a flowchart E detailing how confirmation bias is applied to the user's score. Confirmation bias is a component of cognitive bias in which the user who in general maintains good mental and emotional health are expected to maintain their good health through their habits and actions. Similarly users who in general have not so good mental and emotional health will continue to maintain their status quo. When this rule is violated something impactful has happened through their reported experience. To clarify, when the user who typically seems to be happy and maintains good mental health reports a bad experience, the system identifies this negative event and amplifies the effect by reducing the user's wellness score significantly. The deep learning algorithm further identifies this and suggests Next Best Action that the user needs to do to restore his/her mental and emotional health. Converse is also true, i.e. when the user typically is in not so good mental and emotional health reports a good experience, the system identifies this positive event and amplifies the effect by increasing the user's wellness score appropriately. The deep learning algorithm further identifies this and notes the actions performed by the user that has caused this positive experience for the user. The system then can not only recommend this in the future for this user to increase the probability to have a good experience but also to other user's who have a similar profile as this user. This is to achieve Bandwagon effect i.e share the experiences and suggest others to follow. The flowchart E details the steps involved in amplification and de-amplification of the score based on this very concept.

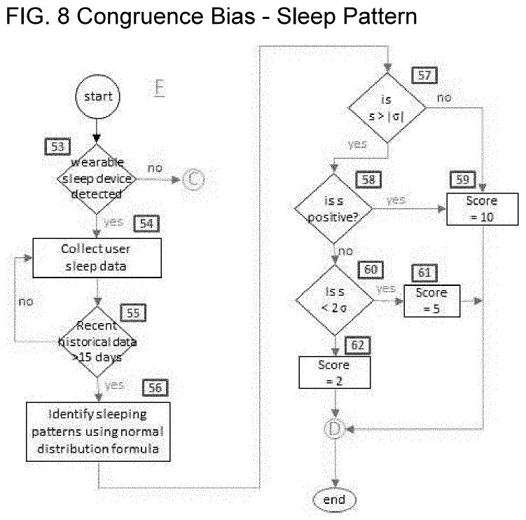

The current score for the user's event 45 is obtained using the steps mentioned in flowchart B referred to in FIG. 4 or from the steps mentioned in flowchart F referred to in FIG. 8. The current color band that the user is assigned is obtained using the steps mentioned in flowchart A referred to in FIG. 3. If the user is in Green or Light Green band 46 and the current score is ten points 47, the user is awarded the current score 50. In case the current score is not ten points, ten points is subtracted from their current score 49. If the user is in Yellow band 48 and their current score is greater than or equal to five points 51, the user is awarded the current score 50. In case the current score is not equal to or greater than five points, five points is subtracted from their current score 52. If the user is not in the Green, Light Green or Yellow band, the user is awarded their current score 50.

Referring to FIG. 8, is a flowchart F explaining in detail how congruence bias is applied to the sleep data retrieved from the user's wearable device 3 referred to in FIG. 1. Congruence bias is a component of cognitive bias where conformity to a principle is strictly followed. The principle here is the user's sleep pattern. If we observe a user's sleep pattern and notice a positive trend over a period of time, the user has conformed to the principle of good development for mental and emotional health. If we observe a user's sleep pattern and notice a negative trend over a period of time, the user has conformed to the principle of not so good development for mental and emotional health. The flowchart F details the steps involved in applying congruence bias to the user's sleep data.

Step 53 checks if the user has a wearable device 3 referred to in FIG. 1. that can collect sleep data on a daily basis 54. In case if the user does not have a wearable device, user's sleep data is obtained through check-in questions using daily check-in method 31 described in flowchart C referred to in FIG. 5. Last fifteen entries of daily sleep data are collected 55 from the user's wearable device 3 referred to in FIG. 1. With normal distribution formula 56 using last fifteen days entries of sleep data population mean μ in sleep hours and standard deviation σ in sleep hours are calculated for this specific user. The variable s stores the sleep hours obtained by subtracting last night sleep hours from the mean μ hours. If the variable s is not greater than one standard deviation (σ) 57, score of ten points 59 is awarded to the user. Otherwise if s hours is a positive number 58, score of ten points 59 is awarded to the user. In case if s hours is a negative number, variable s is then checked again to determine if its less than two standard deviation σ hours 60. If it is, a score of five points 61 is awarded to the user or else score of two points 62 is awarded to the user. The score obtained is then fed to steps mentioned in flowchart D referred to in FIG. 6.

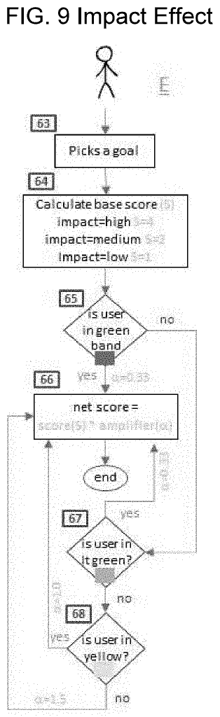

Referring to FIG. 9, is a flowchart E explaining in detail how the user is awarded the points based on the current standing in relation to the wellness (using color bands) and the impact they had for their completed goals. The user picks one of the recommended goals 63. Through conversational method 64 user's impact if they complete the goal is identified. Module 64 also assigns an initial score of four points for high impact, two points for medium and one point for low impact when the user completes the goal. Using the color band assigned to the user in flow chart A referred to in FIG. 3, the assigned score is further amplified or de-amplified. If the user in Green band 65 or in Light Green band 67, the assigned score is de-amplified by multiplying with an amplification factor α of 0.33. In case if the user is in Yellow band 68, the user's score remains the same with the amplification factor α of 1.0. If the user is in any other color band, the user's assigned score is amplified by multiplying with an amplification factor α of 1.5.

The invention claimed is:

1. A wellness scoring system comprising:
a scoring engine running in a user computer device;
a deep learning algorithm implemented in said scoring engine;
a wearable device attached to said scoring engine;
a regularization formula implemented to said deep learning algorithm.

2. A wellness scoring system as in claim 1 wherein:
said deep learning algorithm used to find a color band and a baseline score for all first time users;
a wellness score is established using said baseline score;

3. Using a wellness scoring system as in claim 1 wherein:
a new wellness is obtained by adding said wellness score as in claim 2;
a negativity bias score is obtained using said regularization formula when users check-in to said wellness scoring system on a daily, weekly and monthly basis;
a confirmation bias score is obtained using said confirmation bias formula on said negativity bias score;

4. A wellness scoring system as in claim 1 wherein:
a sleep pattern score is obtained using congruence bias scoring approach on said wearable device;
an impact score is obtained by using next best action recommendations provided in said deep learning algorithm in claim 1;
a total wellness score is obtained by adding, said wellness score in claim 2, said confirmation bias score in claim 3, said impact score, and said sleep pattern score.
